(19) United States
(12) Patent Application Publication
(10) Pub. No.: US 2026/0204343 A1
(43) Pub. Date: Jul. 16, 2026

(54) TARGET RECOMMENDATION FOR DRUG DISCOVERY USING A META-MODEL MACHINE LEARNING SYSTEM

(71) Applicant: SB Technology, Inc., Tarrytown, NY (US)

(72) Inventors: Zane Beckwith, Durham, NC (US); Andrea Bortolato, Metuchen, NJ (US); Jordan E. Crivelli-Decker, El Cerrito, CA (US); Ly Le, Irvine, CA (US); Mary Pitman, Irvine, CA (US); Romelia del Carmen Salomon Ferrer, San Diego, CA (US); Valentin Jean-Baptiste Christian Senicourt, Vancouver (CA); Benjamin Joseph Shields, Candler, NC (US); Lucia Vina Lopez, Santiago de Compostela (ES)

(21) Appl. No.: 19/443,976

(22) Filed: Jan. 8, 2026

Related U.S. Application Data
(60) Provisional application No. 63/746,139, filed on Jan. 16, 2025.

(51) Int. Cl. G16B 15/30; G16B 40/20

(52) U.S. Cl. CPC G16B 15/30; G16B 40/20

(57) ABSTRACT

A method of ranking a plurality of target candidates for a ligand of interest includes calculating first binding affinities for at least a first portion of the plurality of target candidates based on (i) structural information about the plurality of target candidates and (ii) free energy calculations for one or more molecular poses of interest. The method also includes predicting, using a machine learning model, potency values, second binding affinities, or both for at least a second portion of the plurality of target candidates with the ligand. The method also includes generating a list of recommended targets by querying a knowledge graph. The method also includes computing scores for the plurality of target candidates by integrating two or more of the estimated first binding affinities, the predicted potency values, the predicted second binding affinities, or the generated list of recommended targets.

### CROSS-REFERENCE TO RELATED APPLICATION

This application claims priority to U.S. Provisional Patent Application No. 63/746,139, filed January 16, 2025, the entire contents of which are incorporated herein by reference.

### TECHNICAL FIELD

This specification relates to computational approaches for streamlining drug discovery.

### BACKGROUND

Target identification is a step in the drug discovery process that involves the description of specific molecules and/or characterization of biological signaling pathways that can be modulated by the development of new medicines or repurposing of existing drugs. However, target identification techniques often rely extensively on expensive and slow experimental validation from various experimental biology techniques.

### SUMMARY

The present document discloses apparatuses, systems, and methods related to drug discovery and target identification that utilize a machine learning meta-model to combine various techniques including physics-based approaches, proteochemometric modeling approaches, and knowledge graph approaches.

Using existing approaches to target identification, scientific literature data regarding biological signaling pathways, previous drug candidates, structural biology data, and more, are not typically integrated together to help inform the initial target selection in drug discovery programs. Furthermore, existing approaches often do not effectively integrate computational chemistry calculations with other sources of data.

The technology described in this document includes a first-in-class target identification prediction technique with applications in drug discovery, and solves a number of limitations of existing tools.

In one aspect, a method of ranking a plurality of target candidates for a ligand of interest is featured. The method includes calculating first binding affinities for at least a first portion of the plurality of target candidates with the ligand based on (i) structural information about the plurality of target candidates and (ii) free energy calculations for one or more molecular poses of interest. The method also includes predicting, using a machine learning model, potency values, second binding affinities, or both for at least a second portion of the plurality of target candidates with the ligand. The method also includes generating a list of recommended targets by querying a knowledge graph that comprises nodes corresponding to drugs, metabolites, proteins, genes, phenotypes, diseases, or any combination thereof. The method also includes computing scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest.

Implementations can include the examples described below and herein elsewhere. In some implementations, calculating the first binding affinities can include curating protein structures for the plurality of target candidates, extracting bound ligands, predicting binding sites of the plurality of target candidates, and generating the one or more molecular poses of interest. In some implementations, calculating the first binding affinities can include ranking the binding sites of the plurality of target candidates. In some implementations, calculating the first binding affinities can include performing molecular dynamics simulations on the one or more molecular poses of interest. In some implementations, the free energy calculations for the one or more molecular poses of interest can include absolute free energy perturbation calculations. In some implementations, the machine learning model used to predict the second binding affinities can be configured to receive, as input, an amino acid sequence for each of the plurality of target candidates and a SMILES string representing the ligand of interest. In some implementations, predicting the second binding affinities using the machine learning model can include generating encodings representative of the plurality of target candidates and the ligand of interest and predicting the second binding affinities based on the encodings. In some implementations, the machine learning model can include an uncertainty-aware feed forward neural network trained on experimental and in-silico binding affinity values. In some implementations, the knowledge graph can include edges between the nodes, each edge representing a protein-to-protein interaction, a drug-to-protein interaction, or a protein-to-phenotype connection. In some implementations, the nodes of the knowledge graph can include unstructured data including at least one of a small molecule SMILES string, a protein FASTA sequence, a protein 3D structure, text relating to a protein, or a UniProt Protein Accession ID. In some implementations, generating the list of recommended targets can include computing similarity metrics between (i) molecules represented in the knowledge graph and (ii) query molecules selected from the plurality of target candidates. In some implementations, the similarity metrics can be based on at least one of a Tanimoto score, a Log(P) score, a Log(D) score, or small molecule embeddings. In some implementations, generating the list of recommended targets can include generating one or more subgraphs of the knowledge graph, each subgraph including connected edges of the knowledge graph within a k-hop distance to a small molecule entry point, wherein the small molecule entry point is identified based on the similarity metrics. In some implementations, the list of recommended targets can include nodes of the one or more subgraphs that have drug-to-protein edges. In some implementations, the one or more molecular poses of interest can correspond to modifications of the list of recommended targets generated by querying the knowledge graph. In some implementations, generating the list of recommended targets can include determining a similarity between (i) protein interactions represented in subgraphs of the knowledge graph and (ii) observed changes in protein expression after treatment with a bioactive molecule. In some implementations, the protein interactions represented in subgraphs of the knowledge graph can be encoded in graph fingerprints, the observed changes in protein expression after treatment with the bioactive molecule can be encoded in omics fingerprints, and determining the similarity can include computing a similarity metric between the graph fingerprints and the omics fingerprints. In some implementations, the method can also include experimentally assessing the bioactivity, binding affinity, or both of a prioritized subset of the plurality of target candidates with the ligand of interest, wherein the prioritized subset is determined based on the computed scores.

In another aspect, a meta-model machine learning system is featured. The meta-model machine learning system includes a computational chemistry module including computer-executable instructions that, when executed, cause a computing system to calculate first binding affinities for at least a first portion of a plurality of target candidates with a ligand of interest based on (i) structural information about the plurality of target candidates and (ii) free energy calculations for one or more molecular poses of interest. The meta-model machine learning system also includes a proteochemometric module including computer-executable instructions that, when executed, cause the computing system to predict, using a machine learning model, potency values, second binding affinities, or both for at least a second portion of the plurality of target candidates with the ligand. The meta-model machine learning system also includes a knowledge graph module including computer-executable instructions that, when executed, cause the computing system to generate a list of recommended targets by querying a knowledge graph that includes nodes corresponding to drugs, metabolites, proteins, genes, phenotypes, diseases, or any combination thereof. The meta-model machine learning system also includes an integration module including computer-executable instructions that, when executed, cause the computing system to compute scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest. Implementations can include the examples described below and herein elsewhere. In some implementations, calculating the first binding affinities can include curating protein structures for the plurality of target candidates, extracting bound ligands, predicting binding sites of the plurality of target candidates, and generating the one or more molecular poses of interest. In some implementations, calculating the first binding affinities can include ranking the binding sites of the plurality of target candidates. In some implementations, calculating the first binding affinities can include performing molecular dynamics simulations on the one or more molecular poses of interest. In some implementations, the free energy calculations for the one or more molecular poses of interest can include absolute free energy perturbation calculations. In some implementations, the machine learning model used to predict the second binding affinities can be configured to receive, as input, an amino acid sequence for each of the plurality of target candidates and a SMILES string representing the ligand of interest. In some implementations, predicting the second binding affinities using the machine learning model can include generating encodings representative of the plurality of target candidates and the ligand of interest and predicting the second binding affinities based on the encodings. In some implementations, the machine learning model can include an uncertainty-aware feed forward neural network trained on experimental and in-silico binding affinity values. In some implementations, the knowledge graph can include edges between the nodes, each edge representing a protein-to-protein interaction, a drug-to-protein interaction, or a protein-to-phenotype connection. In some implementations, the nodes of the knowledge graph can include unstructured data including at least one of a small molecule SMILES string, a protein FASTA sequence, a protein 3D structure, text relating to a protein, or a UniProt Protein Accession ID. In some implementations, generating the list of recommended targets can include computing similarity metrics between (i) molecules represented in the knowledge graph and (ii) query molecules selected from the plurality of target candidates. In some implementations, the similarity metrics can be based on at least one of a Tanimoto score, a Log(P) score, a Log(D) score, or small molecule embeddings. In some implementations, generating the list of recommended targets can include generating one or more subgraphs of the knowledge graph, each subgraph including connected edges of the knowledge graph within a k-hop distance to a small molecule entry point, wherein the small molecule entry point is identified based on the similarity metrics. In some implementations, the list of recommended targets can include nodes of the one or more subgraphs that have drug-to-protein edges. In some implementations, the one or more molecular poses of interest can correspond to modifications of the list of recommended targets generated by querying the knowledge graph. In some implementations, generating the list of recommended targets can include determining a similarity between (i) protein interactions represented in subgraphs of the knowledge graph and (ii) observed changes in protein expression after treatment with a bioactive molecule. In some implementations, the protein interactions represented in subgraphs of the knowledge graph can be encoded in graph fingerprints, the observed changes in protein expression after treatment with the bioactive molecule can be encoded in omics fingerprints, and determining the similarity can include computing a similarity metric between the graph fingerprints and the omics fingerprints.

In another aspect, a computing system is featured. The computing system includes a memory configured to store instructions and one or more processors configured to execute the instruction to perform operations. The operations include calculating first binding affinities for at least a first portion of the plurality of target candidates with the ligand based on (i) structural information about the plurality of target candidates and (ii) free energy calculations for one or more molecular poses of interest. The operations also include predicting, using a machine learning model, potency values, second binding affinities, or both for at least a second portion of the plurality of target candidates with the ligand. The operations also include generating a list of recommended targets by querying a knowledge graph that comprises nodes corresponding to drugs, metabolites, proteins, genes, phenotypes, diseases, or any combination thereof. The operations also include computing scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest.

Implementations can include the examples described below and herein elsewhere. In some implementations, calculating the first binding affinities can include curating protein structures for the plurality of target candidates, extracting bound ligands, predicting binding sites of the plurality of target candidates, and generating the one or more molecular poses of interest. In some implementations, calculating the first binding affinities can include ranking the binding sites of the plurality of target candidates. In some implementations, calculating the first binding affinities can include performing molecular dynamics simulations on the one or more molecular poses of interest. In some implementations, the free energy calculations for the one or more molecular poses of interest can include absolute free energy perturbation calculations. In some implementations, the machine learning model used to predict the second binding affinities can be configured to receive, as input, an amino acid sequence for each of the plurality of target candidates and a SMILES string representing the ligand of interest. In some implementations, predicting the second binding affinities using the machine learning model can include generating encodings representative of the plurality of target candidates and the ligand of interest and predicting the second binding affinities based on the encodings. In some implementations, the machine learning model can include an uncertainty-aware feed forward neural network trained on experimental and in-silico binding affinity values. In some implementations, the knowledge graph can include edges between the nodes, each edge representing a protein-to-protein interaction, a drug-to-protein interaction, or a protein-to-phenotype connection. In some implementations, the nodes of the knowledge graph can include unstructured data including at least one of a small molecule SMILES string, a protein FASTA sequence, a protein 3D structure, text relating to a protein, or a UniProt Protein Accession ID. In some implementations, generating the list of recommended targets can include computing similarity metrics between (i) molecules represented in the knowledge graph and (ii) query molecules selected from the plurality of target candidates. In some implementations, the similarity metrics can be based on at least one of a Tanimoto score, a Log(P) score, a Log(D) score, or small molecule embeddings. In some implementations, generating the list of recommended targets can include generating one or more subgraphs of the knowledge graph, each subgraph including connected edges of the knowledge graph within a k-hop distance to a small molecule entry point, wherein the small molecule entry point is identified based on the similarity metrics. In some implementations, the list of recommended targets can include nodes of the one or more subgraphs that have drug-to-protein edges. In some implementations, the one or more molecular poses of interest can correspond to modifications of the list of recommended targets generated by querying the knowledge graph. In some implementations, generating the list of recommended targets can include determining a similarity between (i) protein interactions represented in subgraphs of the knowledge graph and (ii) observed changes in protein expression after treatment with a bioactive molecule. In some implementations, the protein interactions represented in subgraphs of the knowledge graph can be encoded in graph fingerprints, the observed changes in protein expression after treatment with the bioactive molecule can be encoded in omics fingerprints, and determining the similarity can include computing a similarity metric between the graph fingerprints and the omics fingerprints.

Various implementations of the technology described herein may provide one or more of the following advantages compared to existing tools and techniques for target identification.

First, the technology described in this document is able to combine multimodal data from heterogenous external and internal knowledge obtained from scientific literature, public databases, and internal data sources.

Second, the technology described herein is able to augment available scientific data with direct computational chemistry simulations for example, binding free energy, molecular docking, and density functional theory calculations.

Third, the technology described herein is able to integrate data from different methodologies (e.g., physics-based approaches, proteochemometric modeling approaches, knowledge graph approaches, etc.) to produce a score that reflects the probability of a given protein (or other target molecule such as RNA or DNA) being a true drug target for a given ligand. This can result in more accurate predictions and rankings of targets compared to existing approaches.

Fourth, the technology described herein can have the advantage of highlighting activity, rather than merely assessing binding affinity. For example, the technology described in this document can integrate information highlighting the likelihood of targets being related to a desired phenotype, enabling more informative prioritization/ranking of active targets.

Fifth, the technology described herein can have the advantage of being widely applicable beyond target identification to a vast set of other drug discovery activities such as drug repurposing and ligand off-target related toxicity.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows a system diagram of a meta-model machine learning system for drug discovery.

FIG. 2 shows a flowchart of operations performed by an example computational chemistry module of a meta-model machine learning system.

FIG. 3 shows a visualization of operations performed by an example computational chemistry module of a meta-model machine learning system.

FIG. 4 shows a flowchart of operations performed by an example proteochemometric modeling module of a meta-model machine learning system.

FIG. 5 shows a visualization of an architecture of an example proteochemometric modeling module of a meta-model machine learning system.

FIG. 6 shows a flowchart of operations performed by an example knowledge graph module of a meta-model machine learning system.

FIG. 7 shows a visualization of a knowledge graph recommendation system.

FIGS. 8, 9, 10A, and FIG. 10B show example results of utilizing a knowledge graph module for target recommendation.

FIG. 11 shows a flowchart of operations performed by an example knowledge graph module of a meta-model machine learning system.

FIG. 12 shows a visualization of an architecture of an example knowledge graph module of a meta-model machine learning system.

FIG. 13 shows example hit enrichment results of a meta-model machine learning system

FIG. 14 shows example hit enrichment results of a proteochemometric modeling module.

FIG. 15 shows example hit enrichment results of a proteochemometric modeling module, a phenotype analysis, and a combination of the two.

FIG. 16 is a flowchart of an example method of ranking target candidates.

FIG. 17 shows an example of a computing device and a mobile computing device that can be used to implement the techniques described here.

Like reference numbers and designations in the various drawings indicate like elements.

### DETAILED DESCRIPTION

Techniques used for drug discovery have limitations. For example, methods focused directly on ligand similarity can be limited in their applicability (e.g., when data about similar ligands is not available). And while methods based on integrating available omics information to infer possible targets for drug discovery can be well-suited to identify targets related to a phenotype, they are limited by the fact that they lack a connection to the ligands.

Many therapeutic candidates are discovered by phenotypic screening where a biological effect is observed, but the target (or targets) that the ligand (e.g., a small molecule) modulates to drive the desired effect may be unknown. In this case, an array of 10s, 100s, 1000s, or more biological macromolecules are screened to determine if they are a binder of the therapeutic of interest and which target binding events drive the desired clinical effect. Likewise, experimental methods may be prohibitively costly, noisy, and have failure modes where common biological targets, such as particular classes of proteins, are systematically mislabeled during the screening campaign.

To successfully identify the targets of a therapeutic (e.g., a ligand) and/or to rank order a set of potential targets, this document describes a meta-model machine learning system (e.g., meta-model machine learning system 102) and related methods that improve upon drug discovery techniques and integrate information (e.g., predictions, rankings, scores, etc.) from multiple techniques (e.g., computational chemistry or physics-based simulations, proteochemometric modeling (PCM) predictions, Knowledge Graph-based techniques, etc.) to overcome their individual limitations. The technology described herein includes a high throughput in silico methodology and corresponding software and systems to accurately rank targets for the binding of a specific drug candidate.

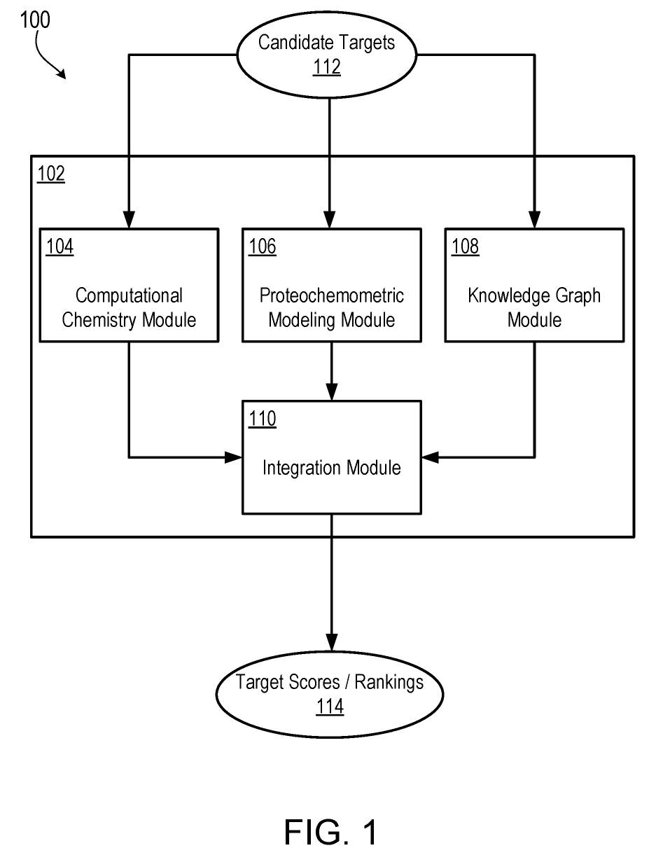

FIG. 1 shows a system diagram 100 of a meta-model machine learning system 102 for drug discovery. The meta-model machine learning system 102 is capable of ranking candidate targets 112 such as proteins based on the likelihood of interacting with one or more given ligands, highlighting the possibility of the candidate targets 112 to be related to a given therapeutic or phenotypic effect. For example, the meta-model machine learning system 102 can receive information about one or more candidate targets 112 and output target scores/rankings 114. The target scores/rankings 114 can be indicative of an expected likelihood that each of the one or more candidate targets 112 interacts with one or more given ligands, and can be used, for example, to prioritize the candidate targets 112 for subsequent exploration. In this way, the meta-model machine learning system 102 can be used to improve and/or accelerate target identification, which is an important part of the drug discovery process. While portions of this document refer to examples where proteins are explored as candidate targets, it is to be understand that in general, alternative classes of target molecules such as RNA or DNA sequences can be identified as candidate targets well. The technology described in this document can also be used and mapped into applications such as phenotypic target identification, toxicity prediction and drug repurposing.

In an example implementation, the meta-model machine learning system 102 includes a computational chemistry module 104, a proteochemometric (PCM) module 106, and a knowledge graph (KG) module 108 that receives information about one or more candidate targets 112 and outputs information such as estimated binding affinities, potency values, lists of recommended targets, rankings of targets, etc. for a ligand of interest. The meta-model machine learning system 102 further includes an integration module 110 that receives the outputs of the individual modules 104, 106, 108 and integrates the information to output target scores and/or rankings 114.

The modules 104, 106, 108, 110 can be implemented via software. They are described, for sake of clear explanation, as distinct components grouped by functionality. However, the modules depicted in the figures are not intended to limit the systems described here to the specific software architectures shown in the figures. In some implementations, one or more of the modules 104, 106, 108, 110 can be separated, combined, or incorporated into a single or combined module.

### Computational Chemistry Module

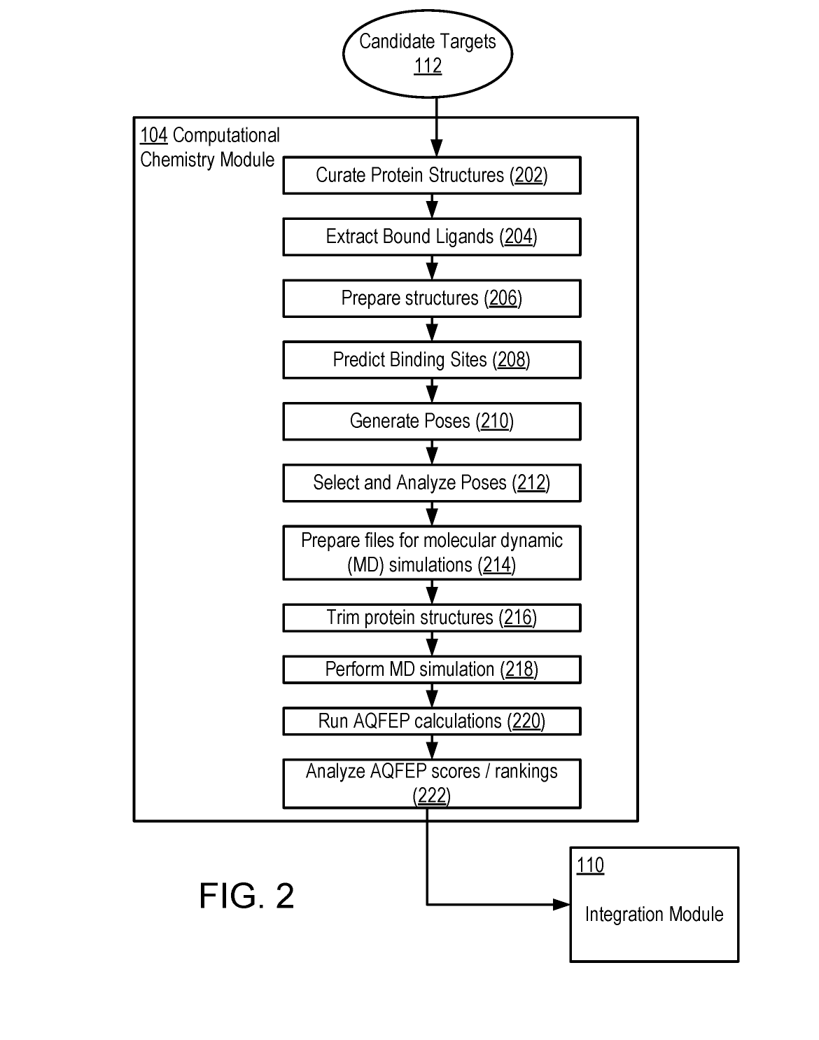

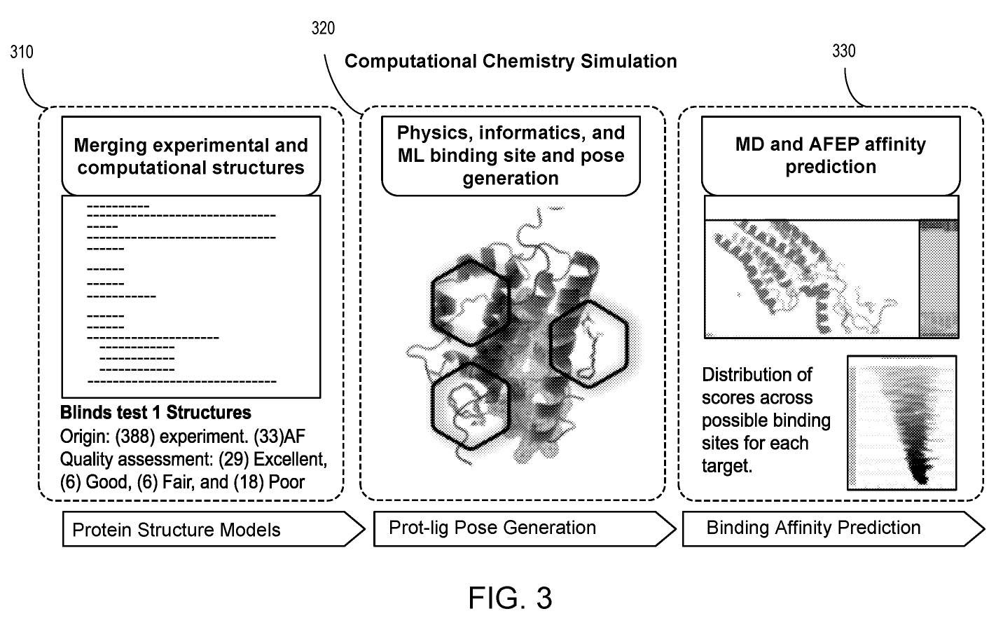

FIG. 2 shows a flowchart of operations performed by an example computational chemistry module 104 of the meta-model machine learning system 102, and FIG. 3 shows a high-level visualization of the operations.

In general, the computational chemistry module 104 is configured to perform a physics-based method to evaluate the binding affinity for each protein of a set of candidate targets 112 via direct calculation. These estimated binding affinities are then used as inputs by the integration module 110.

The physics-based method relies on retrieving available structural information for the proteins being considered. The structural information is analyzed to select the best, most representative structures. This is shown, for example, in block 310 of FIG. 3, representing the merging of experimental and computational structures in a “Protein Structure Models” subprotocol performed by the computational chemistry module 104. As shown in block 310, the computational chemistry module 104 can rank and/or score the structures (e.g., as “excellent”, “good”, “fair”, and “poor”), and select the structures based on this ranking /scoring.

The selected structures are then processed with pocket/pose generation algorithms, including co-folding algorithms, or from homology models derived from KG recommendations (described below), to generate protein-ligand complexes. This is shown, for example, in block 320 of FIG. 3 representing physics, informatics, and ML binding site and pose generation performed by a “Prot-lig Pose Generation” sub-protocol of the computational chemistry module 104.

The generated complexes can be trimmed (e.g., to selected chains or to the binding pocket region) or left whole and evaluated using an absolute free energy method, to estimate binding energy of the potential bioactive molecular pose to each target structure of interest. In some implementations, the absolute free energy method used can be an Absolute Free Energy Perturbation (FEP) process including AQFEP, which can allow lists of up to 100s to 1000s of compounds to be accurately ranked by binding affinity in generally 1-2 T4 GPU hours per compound when measurements upon similar reference systems are not available or otherwise not relied upon for estimating binding affinities. Absolute free energy methods such as AQFEP are described in further detail in U.S. Patent Application No. 18/657,362 (“Multi-Fidelity Free Energy Perturbation Candidate Identification”), which is incorporated by reference herein in its entirety. The estimation of binding energy for the potential bioactive molecular pose to each target structure of interest is shown in block 330 of FIG. 3, representing molecular dynamics (MD) and absolute free energy perturbation (AFEP)-based affinity predictions in a “Binding Affinity Prediction” subprotocol performed by the computational chemistry module 104. Through this process, a distribution of scores across possible binding sites for each target can be determined. The pose for each small molecule with the lowest free energy score (or highest affinity) can then be selected or prioritized for subsequent investigation since it is likely to have greater binding strength. In some implementations, free energy calculations using FEP processes are only implemented upon a subset of candidate targets (e.g., after filtering the candidate targets based on a likelihood of binding success) in order to reduce the search space and computational burden of screening candidate targets using physics-based approaches.

Free energy binding energy calculations are of substantially higher accuracy than docking scores. Therefore, a differentiating factor for the physics-based method performed by the computational chemistry module 104 is the exhaustive retrieval and evaluation of structural information plus the use of a free energy binding affinity method to estimate binding affinities instead of providing a simple docking score. Furthermore, the structural input gathering, filtering, and optimal structural subsetting algorithms performed by the computational chemistry module 104 enables broad and diversified physics-based sampling over target and ligand conformations—at a larger scale than existing techniques—to increase the chances of sampling true complex conformations for affinity predictions and therapeutic mode of action insights.

Referring to FIG. 2, the evaluation of candidate targets 112 by the computational chemistry module 104 can proceed according to the following workflow. First, the computational chemistry module 104 curates protein structures (202) for the candidate targets 212. For example, the protein structure can be curated from protein databases such as RCSB PDB and/or using protein folding tools such as AlphaFold. Second, the computational chemistry module 104 extracts bound ligands (204) and prepares structures (206) for evaluation. Third, the computational chemistry module 104 predicts binding sites (208) for the target candidates 112 based on both known and predicted pockets and then generates poses (210) using a diffusion approach and an empirical scoring function. The computational chemistry module 104 then selects and analyzes the poses (212) and prepares files for molecular dynamic (MD) simulations (213). Next, the computational chemistry module optionally trims the protein structure (216) to only bound chains or to the binding pocket region, and then performs a MD simulation (218) for energy minimization and relaxation. The computational chemistry module 104 then prepares for and runs AQFEP calculations (220) or another absolute free energy method to calculate binding affinity scores, and finally analyzes the AQFEP scores / rankings (222) to assess the most promising of the candidate targets 112.

### Proteochemometric Modeling (PCM) Module

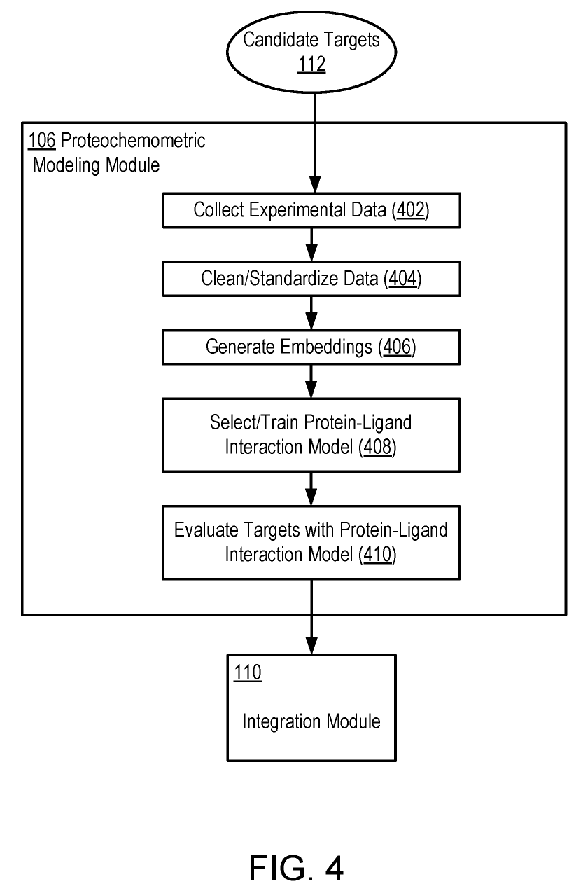

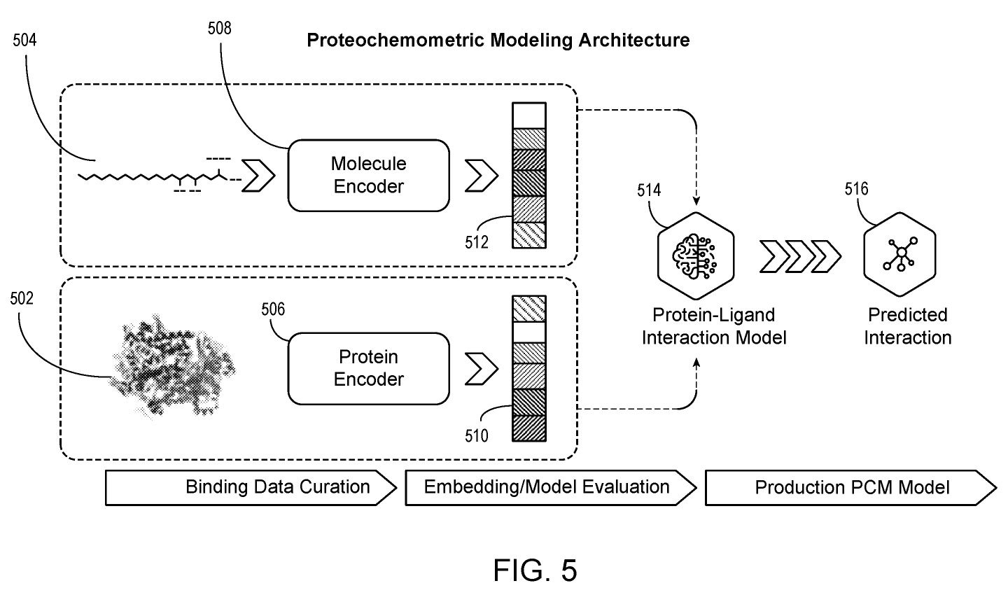

Turning now to the PCM Module 106, FIG. 4 shows a flowchart of operations performed by an example PCM module 106 of a meta-model machine learning system 102, and FIG. 5 shows a visualization of an example architecture of the PCM module 106.

In general, and as shown in FIG. 5, the PCM module 106 implements a machine learning model trained to predict the binding affinity between protein-ligand pairs. This model can take on a number of different architectures (e.g., deep learning architectures, sequence processing architectures, encoding/decoding architectures, etc.) and the best one (or a combination thereof) can be selected based on the specific task at hand. One form of the model, shown in FIG. 5, receives, as input, an amino acid sequence of a target protein 502 and SMILES string representing the target ligand 504. These inputs are featurized into respective embedding vectors 510, 512 by a pretrained protein language model (represented by general protein encoder 506) and ligand-based language model (represented by general molecule encoder 508). These vectors 510, 512 are passed into an uncertainty-aware feed forward neural network (represented by general “Protein-Ligand Interaction Model” 514) which is trained on experimental and in-silico binding affinity values to output a predicted binding affinity, predicted potency / bioactivity, or other predicted interactions (represented by “Predicted Interaction” 516).

An advantage of the architecture just described is that it enables the PCM module 106 to be high throughput, capable of making predictions at a full proteome scale on the order of minutes (e.g., in less than 1 minute, in less than 5 minutes, in less than 10 minutes, in less than 15 minutes, in less than 30 minutes, in less than 60 minutes, in less than 90 minutes, in less than 120 minutes, etc.). However, alternative architectures are envisioned. For example, the inputs to the ML model implemented by the PCM module 106 can be represented in alternative formats other than amino acid sequence or SMILES strings. In some implementations, rather than an embedding model, the molecule encoder 508 and the protein encoder 506 can implement other encoding techniques such as descriptors, fingerprints, or geometric deep learning approaches. And, in some implementations, the protein-ligand interaction model 514 can be a model other than an uncertainty-aware feed forward network, with various supervised learning algorithms available as appropriate substitutes.

Referring to FIG. 4, the evaluation of candidate targets 112 by the PCM module 106 can proceed according to the following example workflow. First, the PCM module 106 collects experimental data (402) or receives such data from a user. For example, the experimental data can include experimental binding affinity data available in databases such as CheMBL or BindingDB. Second, the PCM module 106 cleans and/or standardizes the data (404) and uses the data to generate embeddings (406) for the relevant proteins and ligands. Next, the PCM module 106 selects and trains a protein-ligand interaction model (408) such as the model 514 shown in FIG. 5. Then, the PCM module 106 utilizes the trained protein-ligand interaction model to evaluate targets (410) such as the candidate targets 112. The scores and/or rankings output by the PCM module 106 (e.g., predicted binding affinity values, predicted potency values, etc.) are then provided as inputs to the integration module 110 (described in further detail below). In some implementations, the output of the PCM module can be a predicted potency value (e.g., IC50 value) multiplied by an applicability factor. In some implementations, the applicability factor can be selected to scale the predicted potency value based on a normalized similarity to a particular set of training data. For example, the applicability factor can be computed as a scaled sum of protein sequence similarity value(s) and molecular embedding similarity value(s) of proteins of interest and molecules of interest, respectively, to those that exist in the training data (e.g., with the applicability factor set to 1 if both the protein of interest (the “inference protein”) and molecule of interest (the “inference molecule”) are in the training data set. This technique can have the advantage of counteracting bias in training data sets that are biased toward high potency molecule-protein pairs, which can otherwise result in overprediction of potency values if the inference molecule and/or inference protein are substantially dissimilar from molecules and proteins included in the training data.

While FIG. 4 depicts the PCM module 106 performing data collection and ML training tasks, in some implementations, the PCM module 106 can implement a pre-trained ML model such that operations 402-408 need not be performed for each set of candidate targets 112. Moreover, while FIG. 4 depicts a linear sequence of operations with a single training operation 408, in some implementations, the protein-ligand interaction model can be periodically or continuously trained with additional experimental or in silico data as such data becomes available.

### Knowledge Graph (KG) Module

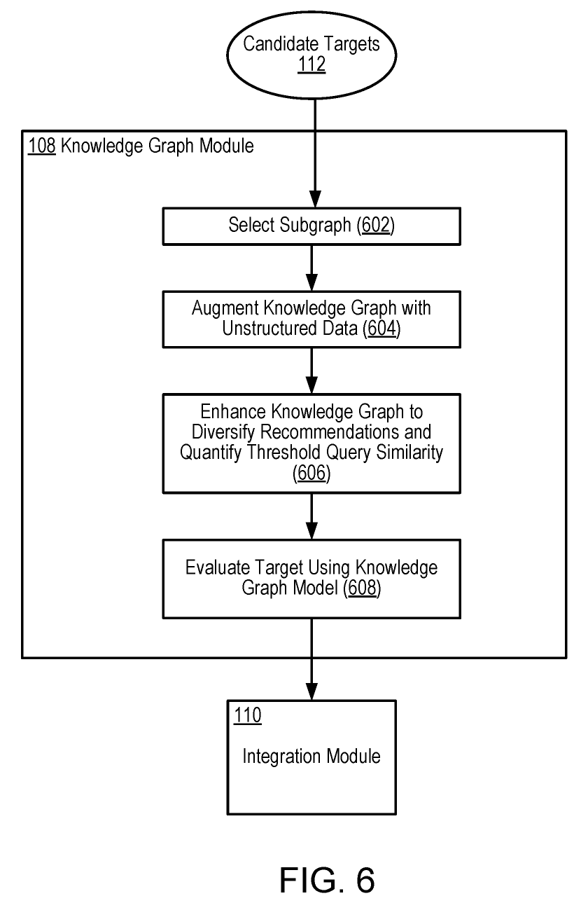

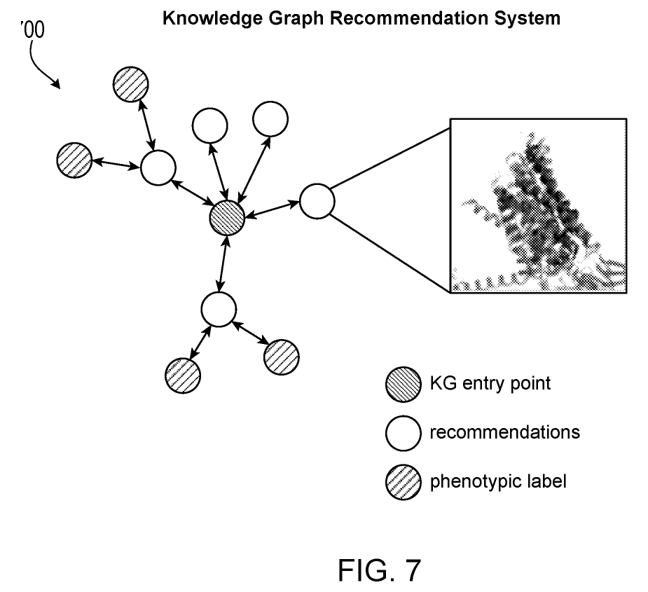

Turning next to the KG module 108, FIG. 6 shows a flowchart of operations performed by an example KG module 108 of a meta-model machine learning system 102, and FIG. 7 shows a corresponding visualization of a knowledge graph recommendation system implemented by the KG module 108. The goal of the KG module 108 in this embodiment, is to use a KG framework to extrapolate quantitatively likely targets based on a ligand query and using recommendation algorithms. The identified targets can be ranked by connection to a phenotype of interest and/or binder promiscuity.

In general, the knowledge graphs (KGs) described herein are composed of (i) nodes representing drugs, metabolites, proteins, genes, phenotypes, diseases, etc. and (ii) labeled edges representing, e.g., protein to protein interactions, drug to protein interactions, protein to phenotype connections, etc. The KGs collect known and validated literature data into an organized database.

The KG solution described in this document and implemented by the KG module 108 includes unstructured data on nodes such as small molecule SMILES strings, protein FASTA sequences, protein 3D structures, informational text on each protein gathered from UniProt (e.g., JSON text), and UniProt Protein Accession IDs. The inclusion of literature data (e.g., from UniProt) and 3D structural information (e.g., experimental, AI, and homology modeling structures) on proteins in the KG is an advancement and helps facilitate new methods of target prediction as described below.

In some implementations, KGs can be queried (e.g., using the KG module 108) based on small molecules of interest to identify similar molecules and the targets to which they bind. For example, a KG such as the KG 700 shown in FIG. 7 can be queried with a compound of interest such that all drugs, small molecules, or metabolites in the KG are compared to the query compound of interest using similarity metrics. These similarity metrics can be customizable and can include a Tanimoto score, a Log(P) score, a Log(D) score, learned and unsupervised small molecule embeddings, or multidimensional similarities based on combinations of any of these measurements. The most similar molecules in the KG to the query molecule are collected (e.g., up to a threshold statistical applicability cutoff that can be set quantitatively based on large scale KG target prediction and refinement or through subject matter expertise on small molecule relevance). This subset of small molecules is then used as entry points into the KG (e.g., the KG entry point shown in FIG. 7) to make subgraphs that include connected edges within a k-hop distance to the small molecule entry point. The subset of nodes with drug-to-protein edges can be taken as a list of recommended targets for the query molecule, ρ (e.g., the recommendations shown in FIG. 7). In addition, this method takes as input a list of query targets that may have been previously prioritized, τ, that can be up to 10³ targets in length or even larger. For each target in ρ and τ, the KG has 3D structural data provided, for example, by a software package that can search for, scrape, download, and filter 100s, 1000s, or 10000s of structures from existing databases and tools. Targets in τ may not exist in the KG (and typically do not). Therefore, in such scenarios, the KG module 108 quantitatively extrapolates likely targets with a novel method explained below to compare the homology and structure of each entry in ρ to each entry in τ.

The quantitative extrapolation of likely targets by the KG module 108 is performed as follows:

First, a KG (e.g., the KG 700) is generated and provided with (e.g., by a software package) with existing or generated experimental, AI-generated, and homology 3D target structures for a particular gene product either in homo sapiens or from other species. For example, the software package can implement an algorithm having tunable parameters to either select the full set of all available structures or it can down-select structures to a reduced set based on filtering criteria. These filtering criteria can be optimized and include calculation of the minimal set of structures for maximal sequence coverage, the minimal number of structures with bound experimental ligands and target coverage, and selection of the single structure per target entry in ρ and τ with the best resolution, sequence length, or presence of a bound small molecule in experimentally resolved structures. Experimentally resolved structures retrieved for a particular gene (e.g., a particular gene UniProt accession code) may contain single chains (homomeric), could be multimeric (disconnected folded polymers), or can contain binding partners such as small molecules, protein fragments, DNA, etc.

Second, in the subset of cases where there is experimental validation that only particular chains are targeted or of interest (e.g., due to experimental data, expert knowledge, etc.) the 3D structures provided to the KG are refined to only include and rank for similarity to the target chains of interest.

Third, an optimal structural alignment of all selected protein chains to all protein chains is performed. For example, this can be performed using a customized version of the USAlign algorithm described by Chengxin Zhang, Morgan Shine, Anna Marie Pyle, and Yang Zhang in US-align: Universal Structure Alignment of Proteins, Nucleic Acids and Macromolecular Complexes. Nature Methods, 19: 1109-1115 (2022). For example, the customized version of the USAlign algorithm can be written in C++, for fast alignment and comparison, which can interface with a Python API interface. In some cases, the customized version of the USAlign algorithm can include customizations to the structure of outputs from the algorithm so that the outputs can be automatically read into Python (e.g., using the “pandas” package) to create dataframes. The alignment algorithm can be used for proteins, DNA, and RNA meaning that this method can be applied to multiple types of macromolecules.

Fourth, the customized USAlign algorithm outputs 3D similarity by root mean square deviation of atomic positions (RMSD), template modeling score (TM-score), number of aligned and similar residues, and number of aligned and similar residues. These metrics are combined algorithmically by the KG module 108 for scoring, with normalization of the cumulative score to 1 over all ρ to τ comparisons. Filtering is performed to remove RMSD values below a threshold RMSD cutoff (e.g., about 0.5 Angstroms which is below the resolution error of typical resolved macromolecular X-ray crystal structures), and to remove total chain lengths below a threshold number of residues (e.g., 5 residues). This filtering step excludes identical chains or small fragments that are experimentally deposited and could result in artificially inflated scores.

Fifth, the top score across target chain alignments per pairwise combinations in ρ and τ for all compared structures is chosen for further ranking integration across recommendations derived from different KG subgraphs.

Sixth, each entry in ρ compared across τ is used to generate a separate target likelihood score.

Seventh, signal analysis is performed to assign a cutoff score value for the top scored (e.g., closest to 1) entries in τ for each ρ either across the set of ρ or for each entry in ρ. This cutoff is customizable based on a KG ranking algorithm optimization over a large learning set to optimize target identification performance, to increase target diversity, for meeting project criteria for numbers of returned predicted targets, or by applying subject matter expertise on target applicability to set a cutoff score.

Eighth, the final list of predicted targets for the small molecule query is returned with the relative target ranks across the sets.

Ninth, in the absence of 3D structural information, or for a faster variant of the algorithm, unstructured data on the KG nodes (SMILES, FASTA sequences) can optionally be compared for similarity across ρ to τ.

Using this method of quantitative extrapolation, the KG module 108 is able to rank candidate targets 112 for a ligand of interest even when those targets do not exist on the knowledge graph. At a high level, referring to FIG. 6, the KG module 108 receives candidate targets 112 and first selects one or more subgraphs (602) defined by the inclusion of certain node types and edge types (as described above). The KG module 108 then augments the KG with unstructured data (604) such as SMILES sequences and 3D structure information, which are added to the nodes. Augmenting the KG can further include altering edge weights (e.g., based on simulations) and edge directions (e.g., to indicate causal relationships). Next, the KG module 108 enhances the KG to diversify recommendations and quantify a threshold query similarity (606). As described above, this can optimize the quality of targets identified via the recommendation and quantitative extrapolation algorithm. Then, the KG module 108 evaluates the candidate targets 112 using the KG model (608). The scores (e.g., promiscuity scores, phenotype scores, etc.), rankings, and/or target recommendations output by the KG module 108 are then provided as inputs to the integration module 110 (described in further detail below).

While FIG. 6 depicts the KG module 108 performing KG selection, augmentation, and enhancement operations, in some implementations, the KG module 108 can implement a pre-defined KG such that operations 602-606 need not be performed for each set of candidate targets 112. Moreover, while KGs are described in this context for use in target identification, in some implementations, KGs could be used in alternative ways such as predicting ligands for targets of interest, performing ensemble-based phenotyping, and predicting protein-to-protein interactions.

In some implementations, KGs can be used to learn additional labels of interest for a target or small molecule list. Example labels of interest include phenotype association scores for targets such as influence on particular disease states, how specific a particular target of interest binds the small molecule of interest (referred to as “promiscuity”), or if nodes in the KG are associated with a class of compounds. Such labels of interest can be derived according to the following example process:

First, additional open-source data is ingested into a KG from one or more databases (e.g., Reactome, OpenTargets, ChEMBL, SureChEMBL, etc.). These open-source databases provide initial quantitative relationships to, for example, phenotypes, for a subset of known proteins. Alternatively, or in addition, for ligand promiscuity, a list of associated chemical language (for example ‘sphingolipid’, ‘nucleic’, etc.) for the query small molecule of interest is generated, e.g., using an LLM.

Second, each node in ρ and τ contains structured data, δ (e.g., JSON data from UniProt and gathered by a software package), which is then ingested to perform a fuzzy match or Retrieval-Augmented Generation (RAG) based search using as input the scores and target labels ingested above. Duplicate appearances of related words or matches in vectors of δ are only counted once and the total length of similarity is tabulated. This algorithm produces a KG-based label score. As an example, for phenotype associations, literature data may indicate the targets X and Y are associated with disease Z. However, the scoring method described herein learns that target B interacts with X and Y by searching and extrapolating from δ and KG connections. Therefore, based on this information, the KG module 108 may predict that target B is also related to disease Z (even if the association was previously unknown).

Third, the known association scores from step one, and the numeric outputs from step two (e.g., the KG-based label scores) are algorithmically combined to produce a final rank for the label of interest.

Fourth, additional labels on the targets or small molecules using this methodology can be applied to further refine or down-select the ranks of targets produced by the KG module 108 when implementing the quantitative extrapolation process described above.

In some implementations, the target recommendations output by the KG module 108 can inform physics-based simulations such as those performed by the computational chemistry module 104. For example, each recommended target can be a known binder of a similar therapeutic drug to the query compound and the complex structures of the targets are typically known and/or retrievable from databases. For high ranking and typically homologous targets output by the quantitative extrapolation process described above, modifications of the recommended target to the target sequence of interest can be performed using open-source homology modeling tools (such as Molecular Operating Environment (MOE)). This can produce a complex that is likely to be in a reasonable binding mode—since the target may be flexible upon therapeutic binding—to associate with the similar query ligand. The coordinates of the experimentally resolved and similar ligand can then be taken as input to guide the query molecule binding mode. The resulting complex structure can serve as a starting pose for physics-based affinity predictions such as those performed by the computational chemistry module 104.

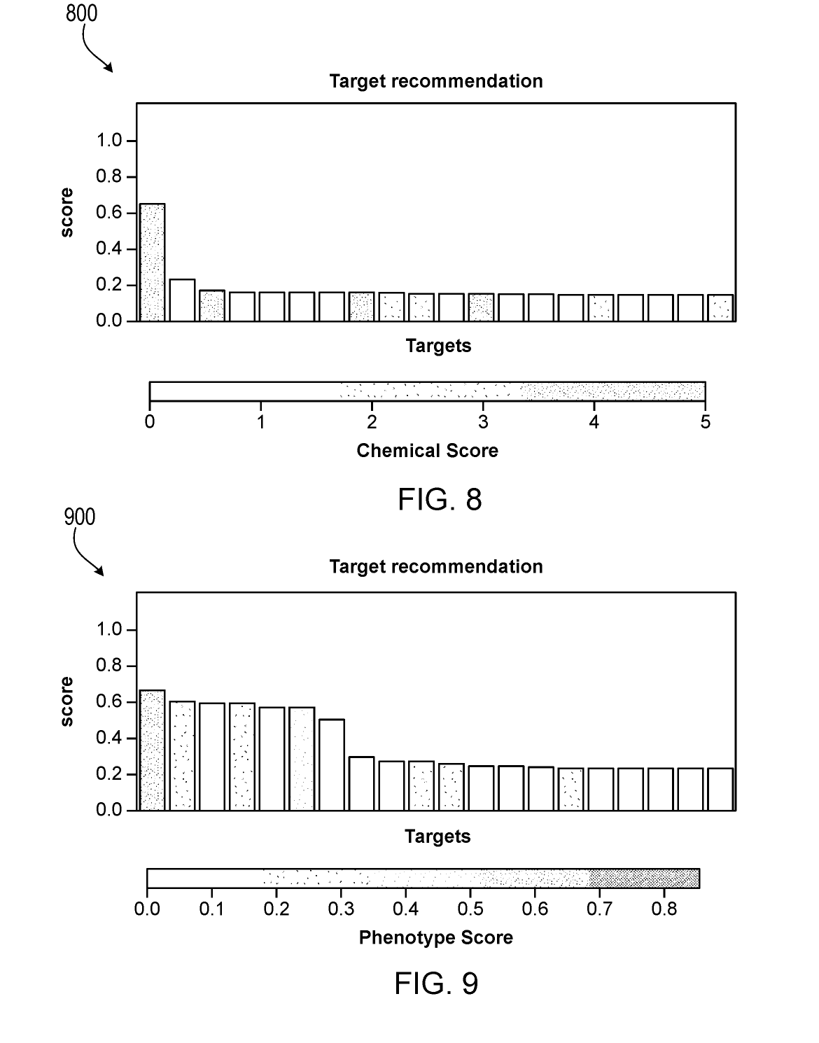

FIGS. 8-10 show example results of utilizing the KG module 108 for target recommendation.

Referring to FIG. 8, a plot 800 is shown in which each bar along the x-axis represents a candidate target for a ligand of interest, and the y-axis values represent binding affinity scores output by the KG module 108. Higher scores along the y-axis indicate a higher predicted likelihood that a ligand of interest binds to the respective candidate target. In addition, the shading of each bar of the plot 800 represents a chemical score output by the KG module 108 for the corresponding candidate target. The chemical score represents a known promiscuity of the candidate target (e.g., determined based on the unstructured node data included in the knowledge graph), with higher chemical scores corresponding to higher promiscuity. In some implementations, multimodal scoring plots such as the plot 800 can be used to filter out or de-prioritize candidate targets as potential false positives. For example, even if a ligand of interest has a high likelihood of binding to a particular target candidate, if that particular target has a high chemical score, it may simply be a promiscuous off-target binder that does not result in potency or bioactivity. Therefore, it can be useful for subject matter experts and/or the integration module 110 to consider both the binding affinity scores and the chemical scores output by the KG module 108.

Referring to FIG. 9, a plot 900 is shown in which each bar along the x-axis again represents a candidate target for a ligand of interest, and the y-axis values represent binding affinity scores output by the KG module 108. Higher scores along the y-axis indicate a higher predicted likelihood that a ligand of interest binds to the respective candidate target. In addition, in the plot 900, the shading of each bar represents a phenotype score output by the KG module 108 for the corresponding candidate target. The phenotype score represents a candidate target’s known potential links to a particular phenotype (e.g., a disease), with darker shading corresponding to a stronger known connection to the phenotype. This phenotype score can be generated by the KG module 108, for example, by performing fuzzy matching or RAG on the unstructured node data included in the knowledge graph, as described above. In some implementations, multimodal scoring plots such as the plot 900 can be used to prioritize candidate targets that have both high binding affinity scores and known links to a phenotype of interest since these candidates are more likely to be true positives for bioactive targets. On the other hand, it is important to note that low phenotype scores may not always represent target candidates that are false positives, but may instead be indicative of a newly found relationship that has not been extensively explored in the scientific literature represented in the knowledge graph. In any case, it can be useful for subject matter experts and/or the integration module 110 to consider both the binding affinity scores and the phenotype scores output by the KG module 108 when performing drug discovery tasks.

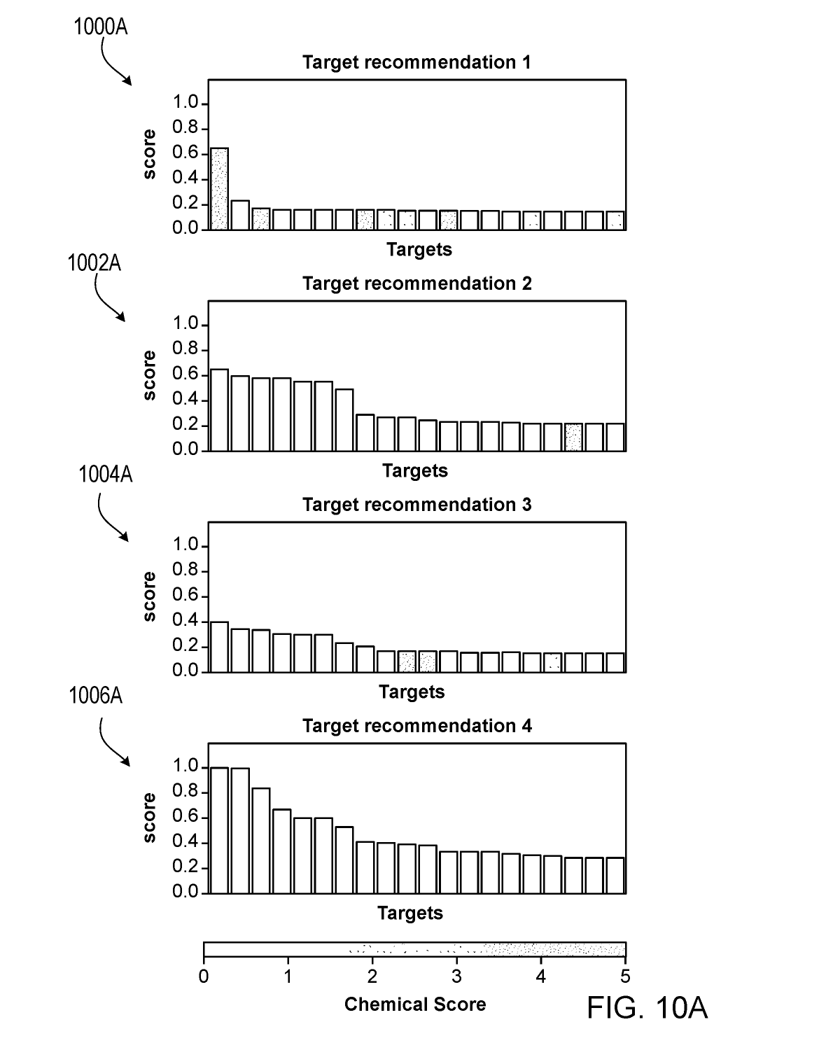

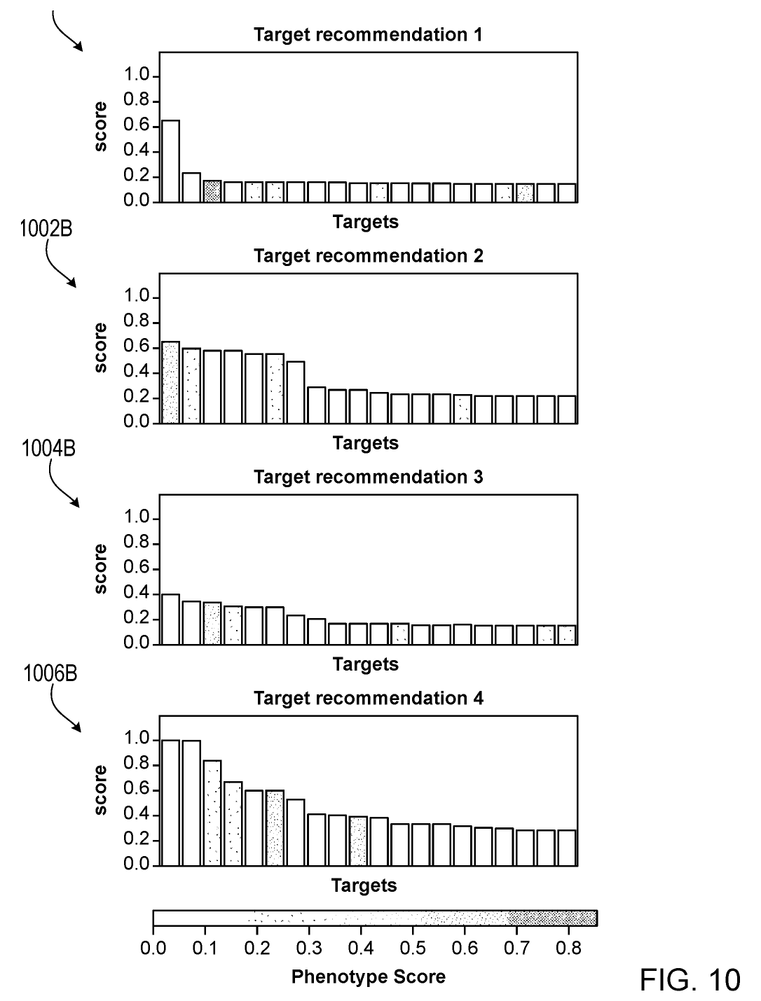

Importantly, while the plot 800 and plot 900 each represent multimodal scores from a single KG subgraph (albeit different subgraphs from each other), similar scores can be computed and plotted for multiple KG subgraphs derived from multiple KG entry points. This can be useful, in some cases, to consider multiple similar ligands of interest. For example, referring to FIGS. 10A and 10B, plots 1000A, 1002A, 1004A, 1006A represent multimodal scoring plots (including chemical scores) for four distinct KG subgraphs, and plots 1000B, 1002B, 1004C, 1006D represent multimodal scoring plots (including phenotype scores) for the same set of four KG subgraphs. In some implementations, the information from these separate subgraphs can be combined into a single collated set of relevant target identification information, and in some implementations, filtering can be implemented (e.g., using a signal cutoff based on a delta in the heights of consecutive bars) before collating the data from the plurality of subgraphs. In this way, the KG module 108 can be utilized for target identification using a multimodal scoring approach.

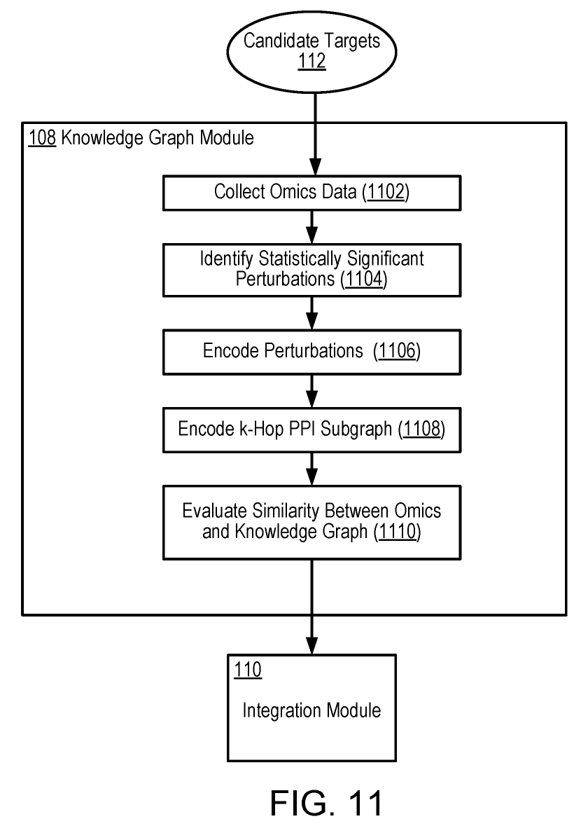

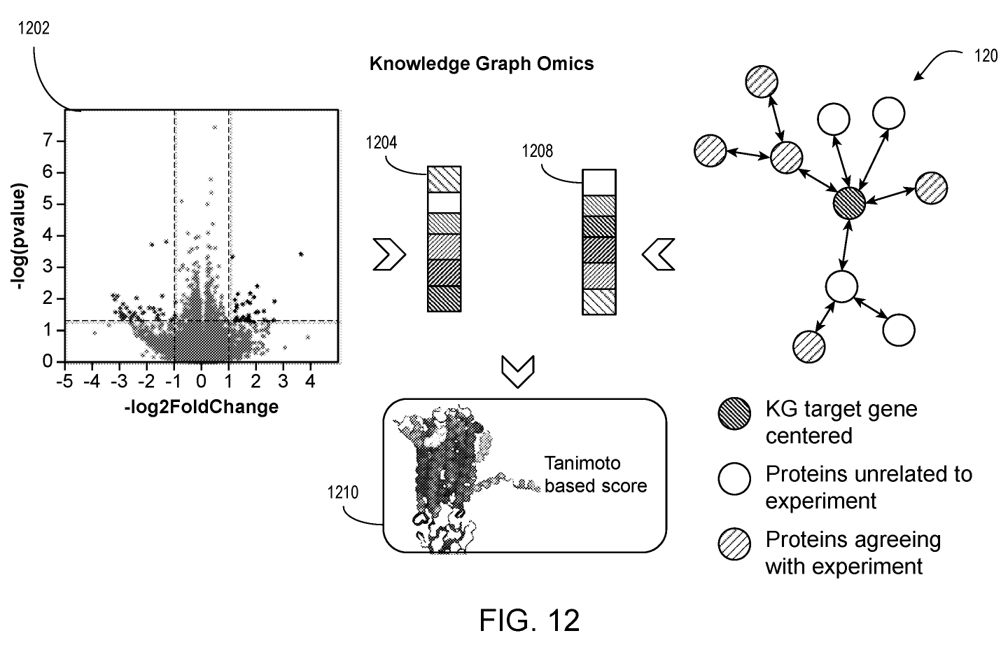

In some implementations, the KG module 108 can also be used in conjunction with omics data to generate target recommendations, scores and/or rankings. FIG. 11 shows a flowchart of operations performed by an example KG module 108 of a meta-model machine learning system 102 configured to utilize omics data, and FIG. 12 shows a visualization of a corresponding architecture of the KG module 108.

Under this approach, observed perturbations in omics experiments (e.g., transcriptomics, proteomics, etc.) with and without a molecule of interest are compared to the expected perturbations encoded in a KG in a customizable way. This enables inference about the most likely KG nodes that account for the observed experimental signals. For example, for target identification using proteomics, one can observe changes in protein expression after treatment with a bioactive molecule and compare those changes to those expected based on the protein interaction network represented in the KG (or KG subgraphs). In this way, the KG framework can be used to rank potential protein targets by statistically significant perturbations observed in vitro and/or in vivo.

Referring to FIG. 11, the KG module 108 collects omics data (1102) and identifies statistically significant perturbations (1104) caused by a molecule of interest. This is shown, for example, in the plot of omics data 1202 in FIG. 12. The KG module 108 then encodes the perturbations (1106), for example, to generate a binary gene fingerprint 1204 shown in FIG. 12. Next, the KG module 108 encodes a k-hop PPI (protein-to-protein interaction) subgraph (1108) centered on each gene in the KG to generate binary graph fingerprints. For example, as shown in FIG. 12, the KG module 108 can encode k-hop PPI subgraph 1206 to generate binary graph fingerprint 1208. Next, the KG module 108 evaluates similarities between the omics and knowledge graph (1110), for example, by computing similarity metrics (e.g., a Tanimoto-based score, Kullback-Leibler divergence metric, fingerprint distance matrix, etc.) between the binary gene fingerprint 1204 and one or more binary graph fingerprints 1208. The resulting similarity metric 1210 shown in FIG. 12 can be indicative of the target gene. For example, if the similarity metric is high, then the KG target gene around which the subgraph 1206 is centered can be selected or scored as a likely target.

While FIG. 11 depicts the KG module 108 performing omics data collection, in some implementations, this data can be provided directly to the KG module 108 by a user such that operation 1102 need not be performed. Moreover, while FIG. 11 depicts a linear sequence of operations, in some implementations, the order of the operations can be modified. For example, the operations 1104 and 1106 and the operation 1108 can be interchangeable in their order, or in some cases, can be implemented in parallel to one another.

### Integration Module

Referring back to FIG. 1, the integration module 110 of the meta-model machine learning system 102 receives the outputs of the individual modules 104, 106, 108 and is trained to integrate the information to output target scores and/or rankings 114. For example, the integration module 110 can implement a ML model that is trained on public benchmarks (e.g., ChEMBL) and internal proprietary benchmarks to maximize the ranking performance of known binders vs. decoys. The model receives as inputs various features from the modules 104, 106, 108 such as chemical structures (both 2D and 3D), 3D protein structures, literature data, and scores from the other technologies discussed above. The model then outputs a probability that a given protein is a target of a ligand of interest. In general, the ML model implemented by the integration module 110 can be implemented with supervised learning architecture. Combining the scores in this way allows for tuning of the importance of different target identification methods in an automated and maximally performant fashion. For example, the integration of methods highlighting the ability of a ligand to interact with a protein from different perspectives, allows for the differentiation between binders and active targets. This can enable the application of the technology described herein to a broad range of drug discovery activities (e.g., toxicity assessment, phenotypic screening, etc.) while incorporating information about binding likelihood and/or estimated potency.

Each of the computational chemistry module 104, the proteochemometric (PCM) module 106, and the knowledge graph (KG) module 108 has unique advantages and disadvantages that the integration module 110 can be trained to account for. For example, physics-based affinity predictions made by the computational chemistry module 104 can have several advantages including the ability to generate new synthetic data; to increase knowledge by predicting binding affinity connected to a given pose which can be used to drive hypotheses of mode of action (MOA); and to generate new knowledge of ligand affinities, ranked binding poses for future optimization, and target dynamics. The computational chemistry module 104 can also have the advantage of utilizing existing 3D structures (e.g., from open-source databases), generated 3D structures (e.g., AI-generated structures or homology models created in house), or recommended 3D structures (e.g., from high scoring KG complex information). The computational chemistry module 104 can further have the advantage of being able to make predictions for any ligand regardless of the existence of previous knowledge, and enabling the investigation of numerous small molecule poses, target conformations, target modifications, and target pockets without the averaging effect of deep learning-based affinity predictions.

The computational chemistry module 104 also has several disadvantages including reliance on the quality of the input structural data. AI-generated structures are known to be lower resolution and to have inaccuracies that can reduce the accuracy of free energy predictions. Therefore, not all target structures may be in the correct conformation to accommodate the ligand binding mode (although leveraging experimental structures with bound ligands can reduce this issue as described above). The computational chemistry module 104 also has inherent uncertainty that a docking or complex prediction protocol generates the lowest energy conformation. Simulations of many poses per target can reduce the impact of this issue but some uncertainty inevitably remains. Physics-based based affinity predictions made by the computational chemistry module 104 can also be slow to generate, taking, for example, multiple days to run absolute binding free energy (ABFE) predictions for many targets and poses due to the data intensive and computationally expensive processes involved. Therefore, physics-based approaches can be applied by the computational chemistry module 104 to the full proteome only with substantial compute investment.

The PCM module 106 has advantages including being a fast method of target prediction that is cheap to train, making it easy to generate binder predictions for 10,000 or more targets. Thus, relative to the physics-based approaches implemented by the computational chemistry module 104, the protocol implemented by the PCM module 106 can more rapidly be applied to the full proteome. The PCM module 106 has additional advantages including being able to be trained on a vast diversity of data to reduce potential bias, being able to be iteratively updated based on prior results or as new training data is available (e.g., experimental and/or simulation-based binding affinity predictions), and being able to predict potency for established protein targets.

Disadvantages of the PCM module 106 include the fact that available training data is imbalanced because the scientific literature is biased towards positive results. Relatedly, since training data is limited to current curated knowledge, the PCM module 106 may struggle to yield accurate predictions for data points far outside of the applicability domain. Furthermore, predictions outputted by the PCM module 106 may not be as interpretable with respect to the therapeutic drivers of biological efficacy as those produced using other methods.

The KG module 108 also has several advantages. First, it can be applied to make quantitative extrapolations in the low data regime because it relies on a connected network of known small molecule and target associations (among other node and edge types, as described above). Second, the KG module 108 can be queried with experimental data (e.g., omics data), augmented with new synthetic or experimental data, or iteratively updated with each application of target identification. In some use cases, the KG module 108 can further be used as a recommendation system to provide complex structures that can be augmented for physics-based simulations. Moreover, the processes implemented by the KG module 108 are cheap to run, scalable, and yield results quickly (e.g., being applicable to the full proteome on the order of hours). Additionally, the KG module 108 can have the advantage of outputting high signal target predictions, rapidly generating additional target or small molecule labels such as phenotype associations (as described above), and focusing on therapeutic drivers with biological efficacy (or “actives”) represented in the knowledge graph.

Some disadvantages of the KG module 108 are as follows. If there is no applicable or similar information within the KG to a particular query, there could be no high signal predicted targets, or a user could select inapplicable subgraphs leading to faulty results. Furthermore, while KGs can be supplemented with 3D structural data (without bias towards experimental, AI-generated, or homology models), if structures of reasonable confidence do not exist or cannot be folded, the intensity of the ranking signal from the KG module 108 can be reduced. In addition, if the KG module 108 is implemented with 3D structural information, the comparison algorithm(s) implemented by the KG module 108 (as describe above) can be slow, taking minutes to hours to run for a single query.

By incorporating information from all of the modules 104, 106, 108, the integration module 110 is able to leverage the advantages of the various techniques described above while mitigating the disadvantages of relying on any individual modules alone. For example, by combining physics-based predictions (e.g., slow, but producing novel information), KGs (e.g., high risk, but yielding high reward extrapolations), and PCM (e.g., fast, but producing an averaging effect) the integration module 110 enables the meta-model machine learning system 102 to output a final target consensus list where the potential liabilities of any individual method are reduced. In some implementations, for faster variants of this methodology, the KG and PCM methods can be combined, or the methods presented here can be combined in any permutation. In addition, while the integration module 110 is described herein as implementing a ML model, in other implementations, the integration module can incorporate information from the modules 104, 106, 108 using any number of techniques, including for example, computing a metric based on weighting, adding, and/or multiplying the scores output by the modules 104, 106, 108.

### Example Hit Enrichment Results

An implementation of the meta-model machine learning system 102 described herein was applied on two blinded tests and retrospective tests. The results show that the correct blinded targets were identified as the top hit and variable targets within the top 15% of possible targets. The application was designed to try to identify the true targets for a given ligand within a list of probable proteins (50 proteins and 100 proteins). The goal was to reduce the list by 50%, with a stretch goal of reducing it to 20% of the original list size. The meta-model machine learning system 102 showed a consistent ability to reduce the list of possible targets, identifying the true targets in top positions. Additionally, the meta-model machine learning system 102 was able to distinguish between true active proteins and generic binders. The meta-model machine learning system 102 was further able to highlight important information like toxicity and target promiscuity. High throughput modules of the system 102, including the PCM module 106 and KG-based phenotype prediction using the KG module 108, have been expanded to be applicable to full proteome target evaluation. The results showed high ability to identify the real targets within the top 5% of the full proteome, particularly when used in conjunction.

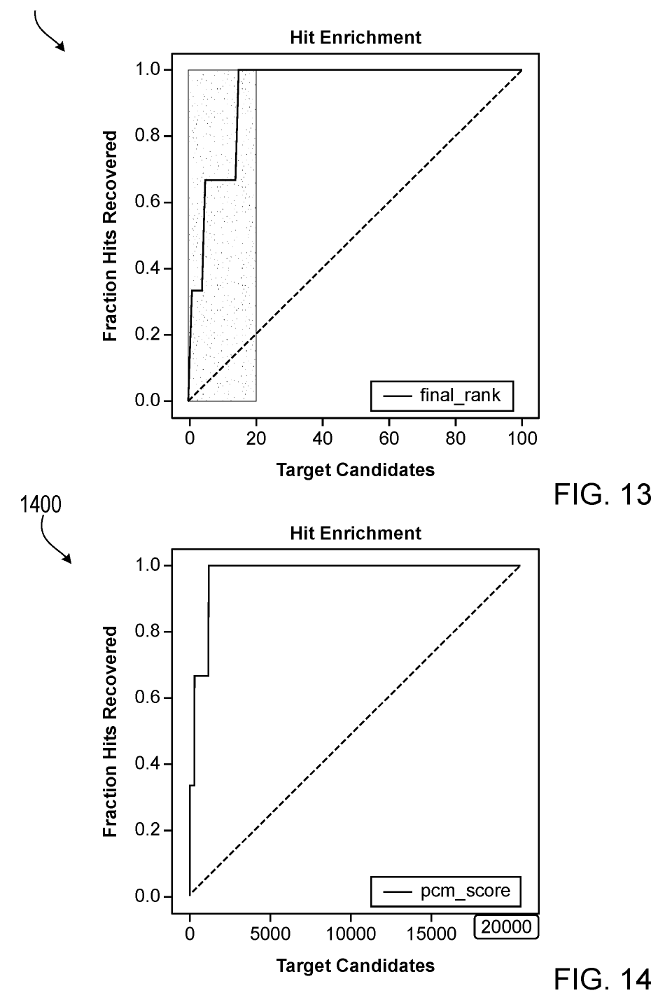

FIG. 13 shows example hit enrichment results of the meta-model machine learning system 102 on a blinded test. In the blinded test, a list of 100 proteins were provided to the meta-model machine learning system 102 as candidate targets, and the meta-model machine learning system 102 provided rankings of the proteins as potential targets without knowing which of the 100 proteins were the true targets. Upon unblinding the study, it was observed that all three of the true targets in the set of 100 proteins were ranked by the meta-model machine learning system 102 within approximately the top 20% of candidate targets (as shown in hit enrichment plot 1300). Notably, the meta-model machine learning system was able to identify targets that were missed by high throughput experimental technologies such as Proteome Integral Solubility Alteration (PISA) and Photoaffinity labeling (PAL). These results highlight the importance and ability of the meta-model machine learning system 102 to identify biological activity over generic binding in order to distinguish known actives from targets with non-specific inhibition. Furthermore, annotations and binding information (e.g., included in the KG of the KG module 108) can be used to identify toxicity and target promiscuity.

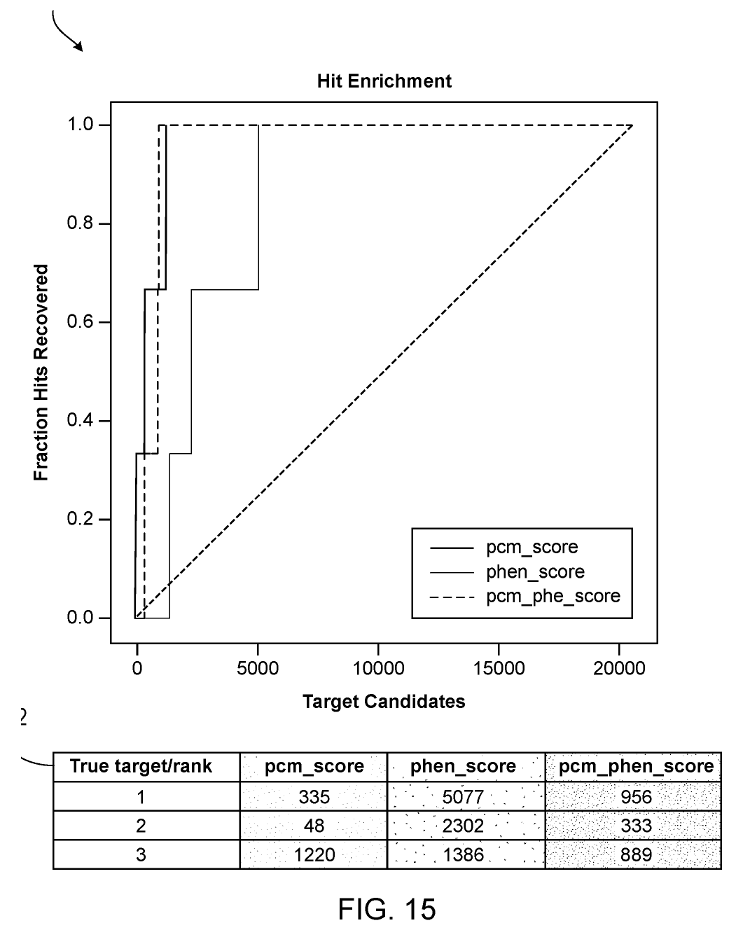

Moving beyond a list of just 100 proteins, the technology described herein can also be used to perform target identification across the whole proteome, including tens of thousands of candidate targets. FIG. 14 shows example hit enrichment results of a PCM module 106 (selected because of its rapid performance) on a full proteome of 20,000 proteins. As shown in the hit enrichment plot 1400, the PCM module 106 alone was able to rank all of the true targets within the top 15% of all possible target, while simultaneously being fast and robust enough to be applied across the whole proteome. Similar methodologies can be used for applications ranging from drug repurposing to toxicity prediction to selectivity analysis. Turning now to FIG. 15, yet another enrichment plot 1500 is shown, this time comparing the performance of the PCM module 106 alone (represented by “pcm_score”), a phenotypic analysis performed by the KG module 108 alone (represented by “phen_score”), and a combination of the two (represented by “pcm_phen_score”) across a list of 20,000 candidate targets. Like the protocol of the PCM module 106, the KG-based phenotype analysis performed by the KG module 108 is fast and can be applied to the full proteome. The phenotype analysis can also be used to help highlight targets that are challenging to identify using the PCM module 106 alone. For example, referring to the table 1502 shown in FIG. 15, the third true target (“True target 3”) was ranked by the PCM module at 1,220out of the 20,000 candidate targets. However, when the KG-based phenotype analysis of the KG module 108 was integrated with the PCM module 106, the third true target was ranked substantially higher (889 out of 20,0000 candidate targets), resulting in all real targets being identified within the top 5% of the full proteome.

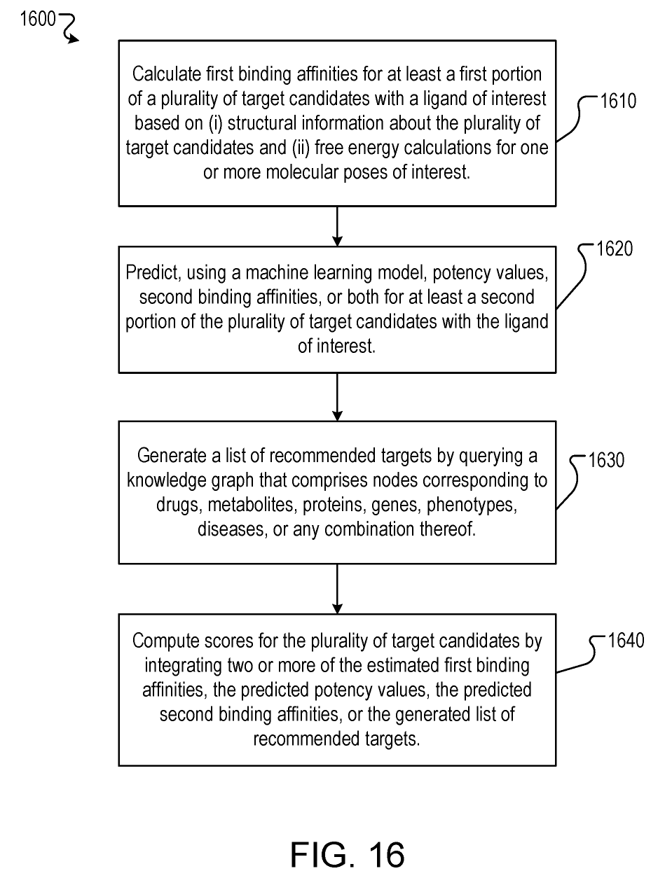

FIG. 16 illustrates an example process 1600 for ranking target candidates (e.g., ranking targets for one or more specific ligands of interest) in accordance with the technology disclosed herein. In some cases, the process 1600 can be performed by the meta-model machine learning system 102 shown in FIG. 1. While the operations of the process 1600 are shown in a particular order, it is to be understood that in various implementations, the operations can be performed in different sequences. For example, the operations 1610, 1620, and 1630 can be interchangeable in their order, or in some cases, can be implemented in parallel to one another.

Operations of the process 1600 include calculating first binding affinities for at least a first portion of the plurality of target candidates with the ligand based on (i) structural information about the plurality of target candidates and (ii) free energy calculations for one or more molecular poses of interest (1610). For example, the operation 1610 can be performed by the computational chemistry module 104, as described above. In some implementations, the operations 1610 can include curating protein structures for the plurality of target candidates, extracting bound ligands, predicting binding sites of the plurality of target candidates, and generating the one or more molecular poses of interest. In some implementations, the operation 1610 can include ranking the binding sites of the plurality of target candidates and/or performing molecular dynamics simulations on the one or more molecular poses of interest. In some implementations, the free energy calculations for the one or more molecular poses of interest can include absolute free energy perturbation calculations such as AQFEP, as described above.

Operations of the process 1600 also include predicting, using a machine learning model, potency values, second binding affinities, or both for at least a second portion of the plurality of target candidates with the ligand (1620). For example, the operation 1620 can be performed by the PCM module 106, as described above. In general, the machine learning model can be any supervised machine learning model. However, in particular implementations, the machine learning model used to predict the second binding affinities can be configured to receive, as input, an amino acid sequence for each of the plurality of target candidates and a SMILES string representing the ligand of interest. In some implementations, the operation 1620 can include generating encodings representative of the plurality of target candidates and the ligand of interest and predicting the second binding affinities based on the encodings. And in some implementations, the machine learning model can include an uncertainty-aware feed forward neural network trained on experimental and in-silico binding affinity values.

Operations of the process 1600 also include generating a list of recommended targets by querying a knowledge graph that comprises nodes corresponding to drugs, metabolites, proteins, genes, phenotypes, diseases, or any combination thereof (1630). For example, the operation 1630 can be performed by the KG module 108, as described above. The knowledge graph can include edges between the nodes, each edge representing a protein-to-protein interaction, a drug-to-protein interaction, or a protein-to-phenotype connection. In some implementations, the nodes of the knowledge graph can include unstructured data including small molecule SMILES strings, protein FASTA sequences, protein 3D structures, text on each protein, and/or UniProt Protein Accession IDs. In some implementations, the operation 1630 can include computing similarity metrics between (i) molecules represented in the knowledge graph and (ii) query molecules selected from the plurality of target candidates. And, in some implementations, the similarity metrics can be based on at least one of a Tanimoto score, a Log(P) score, a Log(D) score, or small molecule embeddings. In some implementations, the operation 1630 can include generating one or more subgraphs of the knowledge graph, each subgraph including connected edges of the knowledge graph within a k-hop distance to a small molecule entry point. The small molecule entry point can be identified based on the similarity metrics, and the list of recommended targets can include nodes of the one or more subgraphs that have drug-to-protein edges. In some implementations, the one or more molecular poses of interest correspond to modifications of the list of recommended targets generated by querying the knowledge graph. In some implementations, the operation 1630 can include determining a similarity between (i) protein interactions represented in subgraphs of the knowledge graph and (ii) observed changes in protein expression after treatment with a bioactive molecule. The protein interactions represented in subgraphs of the knowledge graph can be encoded in graph fingerprints, wherein the observed changes in protein expression after treatment with the bioactive molecule are encoded in omics fingerprints, and wherein determining the similarity comprises computing a similarity metric between the graph fingerprints and the omics fingerprints.

Operations of the process 1600 also include computing scores for the plurality of target candidates by integrating two or more of the estimated first binding affinities, the predicted potency values, the predicted second binding affinities, or the generated list of recommended targets (1640). The computed scores can correlate to at least one of an expected bioactivity, a potency, or a binding affinity of the plurality of target candidates with the ligand of interest. In some implementations, the operation 1640 can be performed by the integration module 110, as described above.

Additional operations of the process 1600 can include the following. In some implementations, the process 1600 can include conducting further experiments to explore an identified target for the purpose of drug discovery. For example, the process 1600 can include experimentally assessing the bioactivity and/or binding affinity of a prioritized subset of the plurality of target candidates with the ligand of interest, wherein the prioritized subset is determined based on the computed scores. In some implementations, the prioritized subset of the plurality of target candidates with the ligand of interest can correspond to a list of target candidates that the meta-model machine learning system 102 (or one of its modules 104, 106, 108) ranks highly.

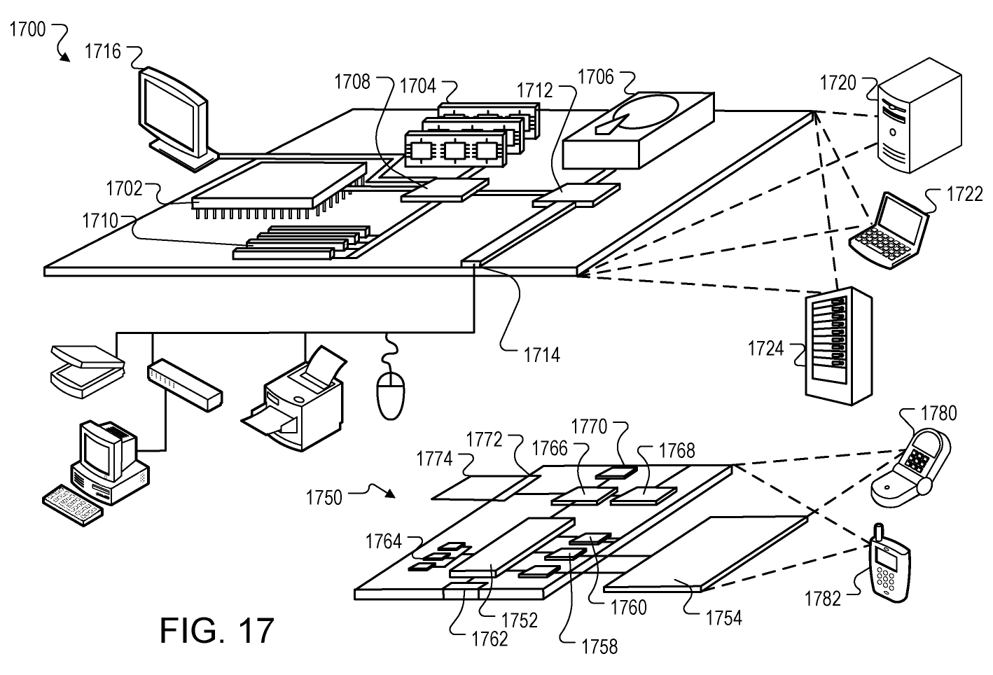

FIG. 17 shows an example of a computing device 1700 and a mobile computing device 1750 that are employed to execute implementations of the present disclosure. The computing device 1700 is intended to represent various forms of digital computers, such as laptops, desktops, workstations, personal digital assistants, servers, blade servers, mainframes, and other appropriate computers. The mobile computing device 1750 is intended to represent various forms of mobile devices, such as personal digital assistants, cellular telephones, smart-phones, AR devices, sensor devices, smart cameras, and other similar computing devices. The components shown here, their connections and relationships, and their functions, are meant to be examples only, and are not meant to be limiting. The computing device 1700 and/or the mobile computing device 1750 can form at least a portion of a computing system that includes the meta-model machine learning system 102. In some implementations, the meta-model machine learning system 102 (including software that implements the machine learning system 102) can be distributed across multiple computing devices 1700 and/or mobile computing devices 1750, for example, to enable distributed computing capabilities. The computing device 1700 and/or the mobile computing device 1750 can be used to perform various operations executed by the meta-model machine learning system 102 and it subcomponents (e.g., the computational chemistry module 104, the proteochemometric modeling module 106, the knowledge graph module 108, and the integration module 110), including but not limited to operations of the process 1600 shown in FIG. 16 (e.g., operations 1610, 1620, 1630, 1640).

The computing device 1700 includes a processor 1702 (e.g., a digital signal processor [DSP], a graphics processing unit [GPU], a field-programmable gate array [FPGA], etc.), a memory 1704, a storage device 1706, a high-speed interface 1708, and a low-speed interface 1712. In some implementations, the high-speed interface 1708 connects to the memory 1704 and multiple high-speed expansion ports 1710. In some implementations, the low-speed interface 1712 connects to a low-speed expansion port 1714 and the storage device 1704. Each of the processor 1702, the memory 1704, the storage device 1706, the high-speed interface 1708, the high-speed expansion ports 1710, and the low-speed interface 1712, are interconnected using various buses, and may be mounted on a common motherboard or in other manners as appropriate. The processor 1702 can process instructions for execution within the computing device 1700, including instructions stored in the memory 1704 and/or on the storage device 1706 to display graphical information for a graphical user interface (GUI) on an external input/output device, such as a display 1716 coupled to the high-speed interface 1708. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 1704 stores information within the computing device 1700. In some implementations, the memory 1704 is a volatile memory unit or units. In some implementations, the memory 1704 is a non-volatile memory unit or units. The memory 1704 may also be another form of a computer-readable medium, such as a magnetic or optical disk.

The storage device 1706 is capable of providing mass storage for the computing device 1700. In some implementations, the storage device 1706 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 1702, perform one or more methods, such as those described above. The instructions can also be stored by one or more storage devices, such as computer-readable or machine-readable mediums, such as the memory 1704, the storage device 1706, or memory on the processor 1702.

The high-speed interface 1708 manages bandwidth-intensive operations for the computing device 1700, while the low-speed interface 1712 manages lower bandwidth-intensive operations. Such allocation of functions is an example only. In some implementations, the high-speed interface 1708 is coupled to the memory 1704, the display 1716 (e.g., through a graphics processor or accelerator), and to the high-speed expansion ports 1710, which may accept various expansion cards. In the implementation, the low-speed interface 1712 is coupled to the storage device 1706 and the low-speed expansion port 1714. The low-speed expansion port 1714, which may include various communication ports (e.g., Universal Serial Bus (USB), Bluetooth, Ethernet, wireless Ethernet) may be coupled to one or more input/output devices. Such input/output devices may include a display device, a printing device 1734, or a keyboard or mouse 1736. The input/output devices may also be coupled to the low-speed expansion port 1714 through a network adapter. Such network input/output devices may include, for example, a switch or router 1732.

The computing device 1700 may be implemented in a number of different forms, as shown in FIG. 17. For example, it may be implemented as a standard server 1720, or multiple times in a group of such servers. In addition, it may be implemented in a personal computer such as a laptop computer 1722. It may also be implemented as part of a rack server system 1724. Alternatively, components from the computing device 1700 may be combined with other components in a mobile device, such as a mobile computing device 1750. Each of such devices may contain one or more of the computing device 1700 and the mobile computing device 1750, and an entire system may be made up of multiple computing devices communicating with each other.

The mobile computing device 1750 includes a processor 1752; a memory 1764; an input/output device, such as a display 1754; a communication interface 1766; and a transceiver 1768; among other components. The mobile computing device 1750 may also be provided with a storage device, such as a microSD card or other device, to provide additional storage. Each of the processor 1752, the memory 1764, the display 1754, the communication interface 1766, and the transceiver 1768, are interconnected using various buses, and several of the components may be mounted on a common motherboard or in other manners as appropriate. In some implementations, the mobile computing device 1750 may include a camera device(s).

The processor 1752 can execute instructions within the mobile computing device 1750, including instructions stored in the memory 1764. The processor 1752 may be implemented as a chipset of chips that include separate and multiple analog and digital processors. For example, the processor 1752 may be a Complex Instruction Set Computers (CISC) processor, a Reduced Instruction Set Computer (RISC) processor, or a Minimal Instruction Set Computer (MISC) processor. The processor 1752 may provide, for example, for coordination of the other components of the mobile computing device 1750, such as control of user interfaces (UIs), applications run by the mobile computing device 1750, and/or wireless communication by the mobile computing device 1750.

The processor 1752 may communicate with a user through a control interface 1758 and a display interface 1756 coupled to the display 1754. The display 1754 may be, for example, a Thin-Film-Transistor Liquid Crystal Display (TFT) display, an Organic Light Emitting Diode (OLED) display, or other appropriate display technology. The display interface 1756 may include appropriate circuitry for driving the display 1754 to present graphical and other information to a user. The control interface 1758 may receive commands from a user and convert them for submission to the processor 1752. In addition, an external interface 1762 may provide communication with the processor 1752, so as to enable near area communication of the mobile computing device 1750 with other devices. The external interface 1762 may provide, for example, for wired communication in some implementations, or for wireless communication in other implementations, and multiple interfaces may also be used.

The memory 1764 stores information within the mobile computing device 1750. The memory 1764 can be implemented as one or more of a computer-readable medium or media, a volatile memory unit or units, or a non-volatile memory unit or units. An expansion memory 1774 may also be provided and connected to the mobile computing device 1750 through an expansion interface 1772, which may include, for example, a Single in Line Memory Module (SIMM) card interface. The expansion memory 1774 may provide extra storage space for the mobile computing device 1750, or may also store applications or other information for the mobile computing device 1750. Specifically, the expansion memory 1774 may include instructions to carry out or supplement the processes described above, and may include secure information also. Thus, for example, the expansion memory 1774 may be provided as a security module for the mobile computing device 1750, and may be programmed with instructions that permit secure use of the mobile computing device 1750. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 1752, perform one or more methods, such as those described above. The instructions can also be stored by one or more storage devices, such as one or more computer-readable or machine-readable mediums, such as the memory 1764, the expansion memory 1774, or memory on the processor 1752. In some implementations, the instructions can be received in a propagated signal, such as, over the transceiver 1768 or the external interface 1762.

The mobile computing device 1750 may communicate wirelessly through the communication interface 1766, which may include digital signal processing circuitry where necessary. The communication interface 1766 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 1768 using a radio frequency. In addition, short-range communication, such as using a Bluetooth or Wi-Fi, may occur. In addition, a Global Positioning System (GPS) receiver module 1770 may provide additional navigation- and location-related wireless data to the mobile computing device 1750, which may be used as appropriate by applications running on the mobile computing device 1750.

The mobile computing device 1750 may also communicate audibly using an audio codec 1760, which may receive spoken information from a user and convert it to usable digital information. The audio codec 1760 may likewise generate audible sound for a user, such as through a speaker, e.g., in a handset of the mobile computing device 1750. Such sound may include sound from voice telephone calls, may include recorded sound (e.g., voice messages, music files, etc.) and may also include sound generated by applications operating on the mobile computing device 1750.

The mobile computing device 1750 may be implemented in a number of different forms, as shown in FIG. 17. For example, it may be implemented a phone device 1780, a personal digital assistant 1782, and a tablet device (not shown). The mobile computing device 1750 may also be implemented as a component of a smart-phone, AR device, or other similar mobile device.

Computing device 1700 and/or 1750 can also include USB flash drives. The USB flash drives may store operating systems and other applications. The USB flash drives can include input/output components, such as a wireless transmitter or USB connector that may be inserted into a USB port of another computing device.

Other embodiments and applications not specifically described herein are also within the scope of the following claims. Elements of different implementations described herein may be combined to form other embodiments.

What is claimed is:

1. A method of ranking a plurality of target candidates for a ligand of interest, the method comprising:
calculating first binding affinities for at least a first portion of the plurality of target candidates with the ligand based on (i) structural information about the plurality of target candidates and (ii) free energy calculations for one or more molecular poses of interest;
predicting, using a machine learning model, potency values, second binding affinities, or both for at least a second portion of the plurality of target candidates with the ligand;
generating a list of recommended targets by querying a knowledge graph that comprises nodes corresponding to drugs, metabolites, proteins, genes, phenotypes, diseases, or any combination thereof; and
computing scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest.

2. The method of claim 1, wherein calculating the first binding affinities comprises curating protein structures for the plurality of target candidates, extracting bound ligands, predicting binding sites of the plurality of target candidates, and generating the one or more molecular poses of interest.

3. The method of claim 2, wherein calculating the first binding affinities further comprises ranking the binding sites of the plurality of target candidates.

4. The method of claim 1, wherein calculating the first binding affinities comprises performing molecular dynamics simulations on the one or more molecular poses of interest.

5. The method of claim 1, wherein the free energy calculations for the one or more molecular poses of interest comprise absolute free energy perturbation calculations.

6. The method of claim 1, wherein the machine learning model used to predict the second binding affinities is configured to receive, as input, an amino acid sequence for each of the plurality of target candidates and a SMILES string representing the ligand of interest.

7. The method of claim 1, wherein predicting the second binding affinities using the machine learning model comprises generating encodings representative of the plurality of target candidates and the ligand of interest and predicting the second binding affinities based on the encodings.

8. The method of claim 7, wherein the machine learning model comprises an uncertainty-aware feed forward neural network trained on experimental and in-silico binding affinity values.

9. The method of claim 1, wherein the knowledge graph comprises edges between the nodes, each edge representing a protein-to-protein interaction, a drug-to-protein interaction, or a protein-to-phenotype connection.

10. The method of claim 1, wherein the nodes of the knowledge graph comprise unstructured data including at least one of a small molecule SMILES string, a protein FASTA sequence, a protein 3D structure, text relating to a protein, or a UniProt Protein Accession ID.

11. The method of claim 1, wherein generating the list of recommended targets comprises computing similarity metrics between (i) molecules represented in the knowledge graph and (ii) query molecules selected from the plurality of target candidates.

12. The method of claim 11, wherein the similarity metrics are based on at least one of a Tanimoto score, a Log(P) score, a Log(D) score, or small molecule embeddings.

13. The method of claim 11, wherein generating the list of recommended targets comprises generating one or more subgraphs of the knowledge graph, each subgraph comprising connected edges of the knowledge graph within a k-hop distance to a small molecule entry point, wherein the small molecule entry point is identified based on the similarity metrics.

14. The method of claim 13, wherein the list of recommended targets comprises nodes of the one or more subgraphs that have drug-to-protein edges.

15. The method of claim 1, wherein the one or more molecular poses of interest correspond to modifications of the list of recommended targets generated by querying the knowledge graph.

16. The method of claim 1, wherein generating the list of recommended targets comprises determining a similarity between (i) protein interactions represented in subgraphs of the knowledge graph and (ii) observed changes in protein expression after treatment with a bioactive molecule.

17. The method of claim 16, wherein the protein interactions represented in subgraphs of the knowledge graph are encoded in graph fingerprints, wherein the observed changes in protein expression after treatment with the bioactive molecule are encoded in omics fingerprints, and wherein determining the similarity comprises computing a similarity metric between the graph fingerprints and the omics fingerprints.

18. The method of claim 1, further comprising experimentally assessing the bioactivity, binding affinity, or both of a prioritized subset of the plurality of target candidates with the ligand of interest, wherein the prioritized subset is determined based on the computed scores.

19. A meta-model machine learning system comprising:
a computational chemistry module comprising computer-executable instructions that, when executed, cause a computing system to calculate first binding affinities for at least a first portion of a plurality of target candidates with a ligand of interest based on (i) structural information about the plurality of target candidates and (ii) free energy calculations for one or more molecular poses of interest;
a proteochemometric module comprising computer-executable instructions that, when executed, cause the computing system to predict, using a machine learning model, potency values, second binding affinities, or both for at least a second portion of the plurality of target candidates with the ligand;
a knowledge graph module comprising computer-executable instructions that, when executed, cause the computing system to generate a list of recommended targets by querying a knowledge graph that comprises nodes corresponding to drugs, metabolites, proteins, genes, phenotypes, diseases, or any combination thereof; and
an integration module comprising computer-executable instructions that, when executed, cause the computing system to compute scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest.

20. A computing system comprising:
a memory configured to store instructions; and
one or more processors configured to execute the instructions to perform operations comprising:
calculating first binding affinities for at least a first portion of a plurality of target candidates with a ligand of interest based on (i) structural information about the plurality of target candidates and (ii) free energy calculations for one or more molecular poses of interest;
predicting, using a machine learning model, potency values, second binding affinities, or both for at least a second portion of the plurality of target candidates with the ligand;
generating a list of recommended targets by querying a knowledge graph that comprises nodes corresponding to drugs, metabolites, proteins, genes, phenotypes, diseases, or any combination thereof; and
computing scores for the plurality of target candidates by integrating two or more of the estimated first binding affinities, the predicted potency values, the predicted second binding affinities, or the generated list of recommended targets, wherein the computed scores correlate to at least one of an expected bioactivity, a potency, or a binding affinity of the plurality of target candidates with the ligand of interest.
